(19) United States
(12) Patent Application Publication
(10) Pub. No.: US 2026/0062554 A1
(43) Pub. Date: Mar. 5, 2026

(54) Stabilized Indigo Carmine Composition

(71) Applicant: Kinnos Inc., Brooklyn, NY (US)

(72) Inventors: Pau OLIVERES, Brooklyn, NY (US); Aaron CHONG, Brooklyn, NY (US); Sara BUSHARAR, Brooklyn, NY (US)

(21) Appl. No.: 19/050,470

(22) Filed: Feb. 11, 2025

Related U.S. Application Data
(63) Continuation of application No. 18/816,268, filed on Aug. 27, 2024, now abandoned.

(51) Int. Cl. C09B 7/02; A61L 2/28; C09B 67/44

(52) U.S. Cl. CPC C09B 7/02; C09B 67/0083; A61L 2/28

(57) ABSTRACT

The present invention provides a composition comprising indigo carmine and a copper (II) metal ion or a salt or hydrate thereof, wherein the composition has a CD value of ≤1.5 and a pH≤5. The present invention significantly increases the shelf-life of aqueous indigo carmine formulations at pH≤5 without requiring special filling or storage conditions to physically remove oxygen from the system. In doing so, aqueous solutions comprising indigo carmine dye are stabilized and suitable for use in, for example, colorized disinfection systems.

### RELATED APPLICATION

This application is a continuation of U.S. application Ser. No. 18/816,268, filed Aug. 27, 2024. The entire teachings of the above application are incorporated herein by reference.

### BACKGROUND

Indigo carmine is a water-soluble dye that is widely known to be susceptible to oxidative degradation in aqueous solutions. This is due to both chemical oxidation by dissolved oxygen in the water as well as photooxidation from direct exposure to sunlight [1]. The dye's aqueous solubility and bright blue hue make it a good candidate for implementation in colorized disinfection systems like the ones taught by Jin et al [5]. Unfortunately, the dye's fast rate of decolorization in aqueous solutions make it difficult to formulate a product with acceptable shelf stability.

Previous attempts to stabilize aqueous solutions of indigo carmine have done so by incorporating pH adjusters and removing oxygen from the composition through physical and chemical means. Umekawa et al [2] teach a stable indigo carmine composition that requires buffering the aqueous dye solution at pH 5-9, adding an oxygen scavenger to the solution, filling under an inert gas atmosphere, and storing in a hermetically sealed container. Similarly, Volkovinskaya et al [3] teach of an aqueous indigo carmine composition that is stabilized by the addition of a sodium citrate pH adjuster as well as rongalite, an antioxidant that reacts irreversibly with dissolved oxygen in solution to consume molecular oxygen and effectively lower its levels in solution.

There is little published literature regarding stabilizing interactions between metal ions and colorants in aqueous solution. Manabe et al [6] teach the use of copper (II) sulfate to stabilize the insoluble, inorganic blue pigment Prussian blue in alkaline solutions above pH 11. At this pH, Prussian blue degrades via interaction with hydroxide anions. Copper (II) sulfate stabilizes the pigment by preferentially reacting with hydroxide ions and forming copper (II) hydroxide. Similarly, Teepakakorn [7] et al teach of a stabilized indigo pigment wherein indigo dye is made into an exceptionally stable insoluble pigment via interactions with Al(III) and Mg(II) ions when adsorbed into the crystalline nano-structures of the mineral perovskite. In both of these cases, the colorants and metal ions are in the solid phase so they cannot be used to create stable aqueous solutions.

The interaction between copper (II) ions and a water-soluble organic dye is discussed by Zanoni et al, who [4] characterized a complex formed by indigo carmine and copper (II) in aqueous solution in a stoichiometric ratio of 1 indigo carmine molecule for every 2 copper (II) ions. This dye-copper complex is reported to only form at pH=7 and above and is only stable for 3 hours after formation.

Therefore, to practically use indigo carmine in aqueous products that need to be shelf stable, it must be stabilized in a manner that does not require extreme measures such as limiting indigo carmine to low concentrations and/or high pH, requiring an oxygen scavenger, and needing to deoxygenate the solutions.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 depicts that the indigo carmine is not stabilized via the formation of complex with the copper (II) metal ions as there is no characteristic absorbance band at 715 nm.

FIG. 2 shows the appearance of a new absorbance peak (UV-VIS peak at 715 nm) with intensity proportional to the amount of copper when the solution is made at pH=10.

### DESCRIPTION OF THE INVENTION

It is an objective of the current invention to provide a significant improvement in the stability of aqueous solutions of indigo carmine dye. In one embodiment of this invention, the stabilized indigo carmine is mixed with solutions of disinfectants to provide transient visible color to surfaces treated with the disinfectant solutions, thus functioning as a disinfection “indicator system”. Furthermore, the stability and antimicrobial efficacy of most disinfectant products is strongly dependent on the pH of the delivered solution. Thus, it's also highly desirable for the dye solutions to not include pH buffering agents.

The present invention significantly increases the shelf-life of aqueous indigo carmine formulations at pH≤5 without the addition of pH adjusters, anti-oxidants, oxygen scavengers, and without requiring special filling or storage conditions to physically remove oxygen from the system. In doing so, aqueous solutions comprising indigo carmine dye are stabilized and suitable for use in, for example, but not limited to, colorized disinfection systems.

The present invention provides a composition comprising indigo carmine and a copper (II) metal ion or a salt or hydrate thereof, wherein the composition has a CD value of ≤1.5 and a pH of ≤5.

It is important for the purpose of clearly describing the various mixtures of indigo carmine and the copper (II) metal ion or salt or hydrate thereof, that fall within the scope of the invention that a new parameter “CD” is defined where:

$$CD = \frac{molar concentration of Cu ( II ) ions in solution}{molar concentration of indigo carmine in solution}$$

A composition having a CD value of 1 is a composition where the amount of indigo carmine and the copper (II) metal ion present in the solution are in equal molar concentrations, i.e., a solution with a 1:1 molar ratio of copper:dye. Compositions with a CD<1 would therefore be those where the molar concentration of the indigo carmine dye in solution is greater than that of copper (II) metal ion. Conversely, compositions with a CD>1 are those in which copper (II) metal ions are present in solution at a higher molar concentration than the indigo carmine dye. Preferably, the composition has a CD value of ≤1. Preferably, the composition has a CD value between 0.008 and 1.5. Preferably, the composition has a CD value between 0.04 and 1. Preferably, the composition has a CD value between 0.1 and 1. Preferably, the composition has a CD value of 0.5.

Preferably, the composition has a pH of <5. The pH of the composition natively lies between 2.5-3.5 but can be optionally adjusted with the addition of an acid, including but not limited to hydrochloric acid, or an alkali, including but not limited to sodium hydroxide, to anywhere in range of pH 0-5.

Copper (II) oxide, or cupric oxide, is an inorganic compound with the formula CuO. Exemplary suitable salts or hydrates of the copper (II) metal ions include, but are not limited to, copper (II) acetate, copper (II) bromide, copper (II) chloride, copper (II) chloride dihydrate, copper (II) cyclohexanbutyrate, copper (II) fluoride, copper (II) fluoride hydrate, copper (II) hydroxide, copper (II) hydroxide phosphate, copper (II) molybdate, copper (II) nitrate, copper (II) nitrate hemi (pentahydrate), copper (II) nitrate hydrate, copper (II) perchlorate, copper (II) pyrophosphate hydrate, copper (II) selenite dehydrate, copper (II) sulfate, copper (II) sulfate pentahydrate, copper (II) tartrate hydrate, copper (II) tetrafluoroborate hydrate, tetraammine copper (II). Preferably, the salts or hydrates of the copper (II) metal ion is selected from copper (II) sulfate, copper (II) sulfate pentahydrate, copper (II) chloride, copper (II) hydroxide, copper (II) nitrate, and copper (II) acetate. Preferably, the salts or hydrates of the copper (II) metal ion is copper (II) sulfate pentahydrate.

The composition of the invention retains exceptional color stability relative to control samples without copper (II) metal ion. The present composition comprising a mixture of indigo carmine and a copper (II) metal ion or a salt or hydrate thereof, combine to make a blue solution of indigo carmine that, even after at least one month when stored at room temperature without the presence of antioxidants and not requiring storage under inert gas or in hermetically sealed containers, retains sufficient blue color.

The use of copper (II) as a stabilizer of aqueous indigo carmine compositions is surprising and unprecedented. Copper (II) is a known catalyst in the Fenton-type oxidative decolorization of organic dyes [8]. Indigo carmine is exceptionally susceptible to decolorization in the presence of copper (II) and oxidants such as peroxides and peroxymonosulfates in aqueous solution and at acidic pH. It is therefore surprising that copper (II) would stabilize indigo carmine against oxidative decolorization from molecular oxygen in acidic aqueous solutions, for example, from pH=0-pH=5 as disclosed herein. Copper (II) is not a known oxygen scavenger the likes of which are taught by Umekawa and Volkoviskaya's stabilized indigo carmine compositions. Copper (II) is not known to react with dissolved oxygen in aqueous solution and there is no evidence that the copper (II) in the present invention is preferentially reacting with oxygen as that would necessarily yield insoluble cuprous and/or cupric oxides that are not found in this composition even after months of storage. This was confirmed by quantitatively measuring the amount of dissolved oxygen of 12 mM indigo carmine solutions with varying concentrations of copper sulfate using a polarographic dissolved oxygen probe. Table 1 shows how, in compositions having a CD values of 0.5, 1, and 2, the concentration of dissolved oxygen in solution is virtually unchanged.

TABLE 1
Concentration of Dissolved Oxygen in different CD formulations

| Formulation | Dissolved Oxygen (ppm) |
|---|---|
| 12 mM Indigo Carmine | 7.4 |
| 12 mM Indigo Carmine + | 7.4 |
| 6 mM Copper(II) Sulfate | |
| CD = 0.5 | |
| 12 mM Indigo Carmine + | 7.3 |
| 12 mM Copper(II) Sulfate | |
| CD = 1 | |
| 12 mM Indigo Carmine + | 7.3 |
| 24 mM Copper(II) Sulfate | |
| CD = 2 | |

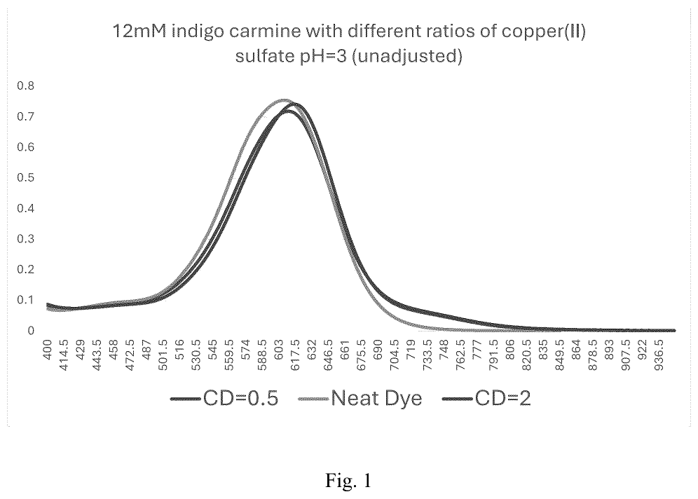

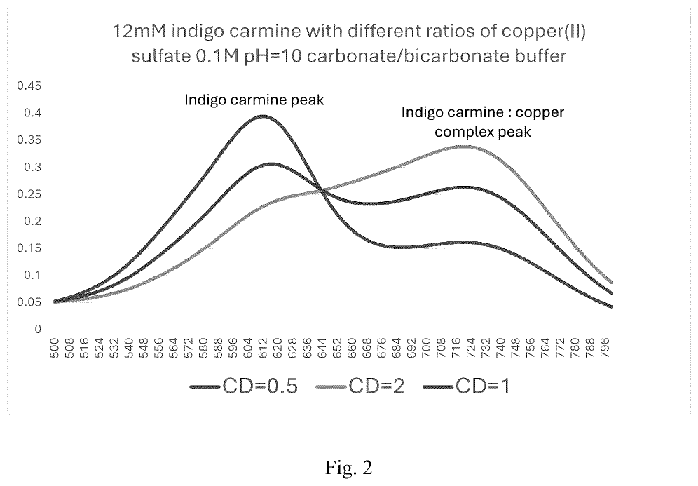

Preferably, the indigo carmine and copper (II) metal ion do not form a complex in the composition. While it is known that metal complexation with dyes can affect dye stability and reactivity, the present invention does not show evidence of the presence of an indigo carmine: copper (II) complex as characterized by Zanoni [4]. Those skilled in the art will realize that the concentration of indigo carmine and complexes between it and metal ions such as copper can be measured via spectrophotometry. FIG. 1 compares the spectrum of an aqueous solution of indigo carmine itself and that of indigo carmine and copper sulfate at molar ratios of 1 indigo carmine molecule: 2 copper (II) ions (CD=2) as well as 2 indigo carmine molecules: 1 copper (II) ions (CD=0.5). The presence of copper in the solution does not create a new peak in the absorbance spectrum at acidic pH. In contrast, FIG. 2 shows the appearance of a new absorbance peak with intensity proportional to the amount of copper when the solution is made at pH=10. The complex exhibits a unique UV-VIS peak at 715 nm. The main peak in the spectrum of indigo carmine is found at 610 nm. The analysis of the spectroscopic data shows that the composition of the complex between indigo carmine and copper ions consists of two copper ions and one indigo carmine molecule, which can be expressed as a copper:dye ratio of 2:1. The pH of the present invention is too low (pH<=5) for the complex described by Zanoni to form in solution. The spectra in FIG. 1 corroborate that the indigo carmine dye in the composition is not stabilized via the formation of complex with the copper (II) metal ion (as in Zanoni) as there is no characteristic absorbance band at 715 nm. The spectrum of the inventive composition is obtained at an indigo carmine concentration of 1.2×10⁻² M (mole/liter). The dye concentration in the spectra reported by Zanoni is much lower, at 1×10⁻⁴ M. Those skilled in the art will realize that obtaining the spectra of the inventive compositions at much higher concentration would increase the sensitivity to detection of the absorption peak due to the complex, and the lack of this peak is consistent with the absence of the complex.

The molar ratio of dye to copper (II) ions in solution should be chosen carefully to ensure that there is enough copper (II) to noticeably decrease the rate of degradation of dye in solution but not so much copper that the ionic strength of the solution is sufficiently high as to induce precipitation of the dissolved species. Table 2 shows how the concentration of dye in solution changes over time in the presence of different amounts of copper. Kinetic studies show that indigo carmine degradation in solution closely matches pseudo-first order kinetics with respect to the dye concentration. In the case of chemical changes in the concentration of a species such as the dye that appear as first order, an equation such as equation 1 can be used to estimate the reaction rate constant, using the absorbance values at 610 nm in the spectrum of the dye solution. Decreases in the dye concentration in solution due to physical instability (precipitation of the dye) or chemical reactions of the dye will be detected by changes in the spectra. Improved stability of the compositions will result in decreases in the reaction rate constants calculated.

$$k = \frac{1}{t} ln ( \frac{{A}_{0}}{{A}_{t}} )$$

### Equation 1: Expression for First-Order Rate Constant k where A₀ and Aₜ are the Concentration of Dye in the Sample at Times 0 and t, Respectively

Table 3 shows the calculated rate constants for the degradation of dye at room temperature in the presence of different amounts of copper, as well as the ratio of different rate constants to the control rate constant (no copper). CD values of 0.0008 and 0.004 did not meaningfully decrease the rate of decay of dissolved indigo carmine. Increasing the concentration of copper by a factor of 5 from 0.01 mM to 0.05 mM did not meaningfully change the rate of degradation. Increasing the concentration of copper from 0.05 mM to 0.1 mM decreased the value of the degradation rate constant by 13%, a significant amount Thus, a change in the stability of the indigo carmine solution occurs at a CD value between 0.008 and 0.004. At CD values above 0.008 we observed a clear correlation between increased CD and decreased rate of dye degradation.

Note that the concentration of copper (II) metal ions or salts or hydrates thereof in millimoles/liter (mM) may be converted to a concentration in weight percent using the equation:

$$\frac{[ Cu ( II ) SO 4 ] mM}{10000} \times 159.609 g {mol}^{- 1} = \% [ Cu ( II ) SO 4 ] w / v$$

Similarly, the concentration of indigo carmine dye (disodium salt) in millimolar (mM) may be converted to a concentration in weight percent using the equation:

$$\frac{[ Indigo Carmine ] mM}{10000} \times 466.36 g {mol}^{- 1} = \% [ Indigo Carmine ] w / v$$

TABLE 2
Change in indigo carmine concentration over time
at room temperature with different CD values

| | % of original dye remaining of | | | |
|---|---|---|---|---|
| | original 12 mM[0.56% w/v] | | | |
| | indigo carmine solution | | | |
| | after X days at room temperature | | | |
| | Day 0 | Day 11 | Day 17 | Day 28 |
| No copper | 100% | 82% | 74% | 59% |
| 0.01 mM anhydrous | 100% | 83% | 75% | 59% |
| Cu(II) sulfate | | | | |
| [0.00016% w/v] | | | | |
| (CD = 0.0008) | | | | |
| 0.05 mM anhydrous | 100% | 88% | 78% | 59% |
| Cu(II) sulfate | | | | |
| [0.0008% w/v] | | | | |
| (CD = 0.004) | | | | |
| 0.1 mM anhydrous | 100% | 88% | 82% | 64% |
| Cu(II) sulfate | | | | |
| [0.0016% w/v] | | | | |
| (CD = 0.008) | | | | |
| 0.5 mM anhydrous | 100% | 94% | 92% | 81% |
| Cu(II) sulfate | | | | |
| [0.008% w/v] | | | | |
| (CD = 0.04) | | | | |
| 1 mM anhydrous Cu(II) | 100% | 97% | 96% | 88% |
| sulfate [0.016% w/v] | | | | |
| (CD = 0.08) | | | | |

TABLE 3
Calculated 1^{st} order rate constant for
degradation of 12 mM[0.56 w/v] indigo carmine
at room temperature with different CD ratios

| | Calculated 1^{st} | Ratio of |
|---|---|---|
| | order rate | copper |
| | constant for room | degradation |
| | temperature | rate to control |
| | degradation | degradation |
| No copper | 0.019 | 1 |
| 0.01 mM anhydrous | 0.019 | 1 |
| Cu(II) sulfate | | |
| [0.00016% w/v] | | |
| (CD = 0.0008) | | |
| 0.05 mM anhydrous | 0.019 | 1 |
| Cu(II) sulfate | | |
| [0.0008% w/v] | | |
| (CD = 0.004) | | |
| 0.1 mM anhydrous | 0.016 | 0.83 |
| Cu(II) sulfate | | |
| [0.0016% w/v] | | |
| (CD = 0.008) | | |
| 0.5 mM anhydrous | 0.0072 | 0.38 |
| Cu(II) sulfate | | |
| [0.008% w/v] | | |
| (CD = 0.04) | | |
| 1 mM anhydrous Cu(II) | 0.0045 | 0.24 |
| sulfate [0.016% w/v] | | |
| (CD = 0.08) | | |

The observed increase in dye stability plateaus after a CD of 0.5. Table 4 shows how there is not a significant difference in dye degradation after 81 days between samples with CD=0.5 and CD=1. This holds true at both the solution's native pH of 3 and if the solution is acidified to pH=1 with 2M HCl, which is a clear advantage of the system's formulation flexibility. A control sample with no copper dropped to 28% of its original dye concentration in that same time.

TABLE 4
12 mM[0.56% w/v] indigo carmine % of original
dye remaining after 81 days at room temperature

| No solubilizers | pH = 1 | pH = 3 |
|---|---|---|
| No Cu* | — | 28% |
| 6 mM[0.096% w/v] | 0.89 | 91% |
| anhydrous Cu(II) | | |
| sulfate (CD = 0.5) | | |
| 12 mM | 0.91 | 90% |
| [0.19% w/v] | | |
| anhydrous Cu(II) | | |
| sulfate (CD = 1) | | |

*Control solution was sampled at t = 76 days. Control was not monitored at pH = 1.

The increased stability of aqueous indigo carmine in the presence of copper (II) has been observed to be independent of dye concentration. Table 5 shows how the degradation of 4 mM indigo carmine at room temperature is significantly slowed down by copper (II) in ratios of CD=1 and CD=0.5.

TABLE 5
4 mM[0.19% w/v] indigo carmine % of original dye
remaining after 7 days at room temperature

| | |
|---|---|
| No Cu | 73% |
| 2 mM Cu(0.032% w/v) | 96% |
| anhydrous Cu(II) sulfate | |
| (CD = 0.5) | |
| 4 mM Cu(0.064% w/v) | 96% |
| anhydrous Cu(II) sulfate | |
| (CD = 1) | |

Improved storage stability at low temperatures is also surprisingly and desirably affected by the presence of copper (II) metal ions or salts or hydrates thereof in the composition. For example, systems at pH less than or equal to 3 formulated at CD>=1 showed formation of visible precipitates when stored for 7 days at 4° C. In contrast, formulations with CD<1 provided stability at temperatures ranging from about 4° C. to room temperature, i.e. about 20° C.

Visual examinations show that the minimum concentration of indigo carmine that will impart bright and visible color is around 6 mM. Preferred compositions therefore include at least 6 mM [0.096% w/v] indigo carmine.

In embodiments, the composition consists essentially of indigo carmine and a copper (II) metal ion or a salt or hydrate thereof, wherein the composition has a CD value of ≤1.5 and a pH of ≤5. In embodiments, the composition consists of indigo carmine and a copper (II) metal ion or a salt or hydrate thereof, wherein the composition has a CD value of ≤1.5 and a pH of ≤5.

Note that while the source of copper (II) ions was chosen to be copper (II) sulfate pentahydrate for this example, the scope of the invention is not limited to a specific salt of copper (II) as the source of copper (II) ions for any given embodiment.

### Optional Adjuvants

A variety of optional adjuvant or mixture of optional adjuvants may be present in the liquid formulation. For example, in some embodiments, the composition of the invention optionally comprises one or more solubilizing agents (also referred to herein as solubilizers) in order to modulate the aesthetics of the liquid solutions. Solubilizers included in formulations of the invention are any acceptable solvent that can be used to increase the solubility of the dye in the presence of the copper (II) metal ions or a salt or hydrate thereof. Examples of solubilizing agents include, but are not limited to, glycol ethers, alcohols, diols and glycols. The glycol ethers may include, but are not limited to, C₁₋₁₀ alkyl ethers of alkylene glycols and polyalkylene glycols. Preferably, solubilizers include, but are not limited to, propylene glycol n-butyl ether, tryptophol, tyrosol, phenethyl alcohol (phenylethanol), phenoxyethanol, benzyl alcohol, hydroxy tyrolsol and derivatives thereof. Preferably, the solubilizer is phenethyl alcohol. Preferably, the solubilizer is phenoxyethanol.

The compositions of the present invention may contain surfactants selected from nonionic, anionic, cationic, ampholytic, amphoteric and zwitterionic surfactants and mixtures thereof. The addition of one or more surfactants can increase the apparent solubility of the dye via incorporation of the dye in surfactant micelles. A typical listing of anionic, ampholytic, and zwitterionic classes, and species of these surfactants, is given in U.S. Pat. No. 3,929,678 to Laughlin. A list of suitable cationic surfactants is given in U.S. Pat. No. 4,259,217 to Murphy. When present, so-called “green” surfactants such as, but not limited to rhamnolipids, sophorolipids, or alkyl glucosides are preferred. However, addition of surfactant to the dye solutions is not preferred, in order to maximize the compatibility of the dye solution with a wide range of disinfectant formulations.

The compositions of the present invention may optionally contain perfumes, fragrance or fragrance release agents, pH adjusting agents such as, but not limited to, alkali metal hydroxides, inorganic or organic acids or amino alcohols.

Optionally, antimicrobial preservatives may be present, including, but not limited to, so-called natural materials such as essential oils or extracts, formaldehyde-releasing agents or non-formaldehyde-based preservatives.

Although not preferred, buffers or builders or chelating agents or sequestrants may be present. Examples include, but are not limited to, EDTA salts, GLDA, gluconates, citric acid, 2-hydroxy acids and glutamic acid and derivatives.

Other adjuvants that may be present in effective amounts include rheology modifiers or thickeners and ingredients to stabilize the liquid solutions such as antioxidants, including, but not limited to, so-called natural materials such as retinol or other vitamins or pro-vitamins, cloud-point modifiers, or hydrotropes.

The invention is further illustrated by the following examples, which should not be construed as further limiting. The practice of the present invention will employ, unless otherwise indicated, conventional techniques which are within the skill of the art.

### Example 1: Formulating a Composition with CD=0.5

A composition with CD=0.5 is one where the concentrations of copper (II) and indigo carmine in solution satisfy the condition

$$\frac{molar concentration of Cu ( II )}{molar concentration of indigo carmine} = 0.5$$

In one example of a composition having a sufficient color intensity, the composition comprises an indigo carmine concentration to be 12 mM and, as such, the concentration of Cu(II) metal ion in solution would have to be 6 mM for the composition's CD value to equal 0.5. 1 L of this composition with CD=0.5 would comprise:

$$1000 g of water$$

$$12 m mol of indigo carmine = 0.012 mol indigo carmine = 0.012 mol \times 466.36 g {mol}^{- 1} = 5.6 g indigo carmine$$

$$6 m mol \times 249.685 g {mol}^{-} = 1.5 g copper ( II ) sulfate pentahydrate$$

### REFERENCES

1. J Ristea (Sep. 20 2023) Indigo Carmine: Between Necessity and Concern, Xenobiot. 2023 September; 13 (3): 509-528. doi: 10.3390/jox13030033.
2. WO2010018723A1.
3. Russian patent SU136852.
4. Zanoni 2010, “Exploratory Study on Sequestration of Some Essential Metals by Indigo Carmine Food Dye”.
5. U.S. Pat. No. 11,464,371B2.
6. Manabe et al. (2020) Stabilization of Prussian blue using copper sulfate for eliminating radioactive cesium from a high pH solution and seawater Journal of Hazardous Materials Volume 386, 121979, https://doi.org/10.1016/j.jhazmat.2019.121979.
7. Teepakakorn et al. (2019). The Improved Stability of Molecular Guests by the Confinement into Nanospaces. Chemistry Letters.
8. Bokare and Choi, Review of Iron-Free Fenton-like Systems for Activating H₂O₂ in Advanced Oxidation Processes.

While this invention has been particularly shown and described with references to preferred embodiments thereof, it will be understood by those skilled in the art that various changes in form and details may be made therein without departing from the scope of the invention encompassed by the appended claims.

1. A stabilized indigo carmine composition comprising indigo carmine and a copper (II) metal ion or a salt or hydrate thereof, wherein the composition has a CD value of from 0.04 to 1.5 and a pH of ≤5.

2. The composition according to claim 1, wherein the copper (II) metal ion is in the form of a salt.

3. The composition according to claim 1, wherein the copper (II) metal ion salt is selected from copper (II) sulfate, copper (II) sulfate pentahydrate, copper (II) chloride, copper (II) hydroxide, copper (II) nitrate, and copper (II) acetate.

4. The composition according to claim 3, wherein the copper (II) metal ion salt is copper (II) sulfate pentahydrate.

5. (canceled)

6. The composition according to claim 1, wherein the CD value is between 0.04 and 1.

7. The composition according to claim 1, wherein the CD value is between 0.1 and 1.

8. The composition according to claim 1, wherein the CD value is from 0.1 to 0.5.

9. (canceled)

10. The composition according to claim 1, wherein the composition further comprises a solubilizing agent.

11. The composition according to claim 10, wherein the solubilizing agent is selected from the group propylene glycol n-butyl ether, tryptophol, tyrosol, phenethyl alcohol, phenoxyethanol, benzyl alcohol, hydroxy tyrolsol and derivatives thereof.

12. The composition according to claim 11, wherein the solubilizing agent is phenoxyethanol or phenethyl alcohol.

13. The composition according to claim 1, wherein the composition retains a blue color with at least 80% Indigo carmine remaining after 1 month without the presence of antioxidants and not requiring storage under inert gas or in a hermetically sealed container.
